(19) United States
(12) Patent Application Publication
(10) Pub. No.: US 2021/0322604 A1
(43) Pub. Date: Oct. 21, 2021

(54) PIVOTING LIGHT CAROUSEL FOR USE WITH CLEANING AND/OR DISINFECTING CRADLE FOR VIRTUAL REALITY HEADSETS

(71) Applicant: Cleanbox Technology, Inc., Carlbad, CA (US)

(72) Inventor: David Allen Georgeson, Vista, CA (US)

(21) Appl. No.: 17/272,350

(22) PCT Filed: Aug. 28, 2019
(86) PCT No.: PCT/US19/48637

§ 371 Date: Mar. 1, 2021

Related U.S. Application Data
(60) Provisional application No. 62/724,541, filed on Aug. 29, 2018.
(60) Provisional application No. 62/728,652, filed on Sep. 7, 2018.

(51) Int. Cl. A61L 2/10; F21V 14/02

(52) U.S. Cl. CPC A61L 2/10; F21V 14/02; F21Y 2115/10; A61L 2202/15; A61L 2202/16; A61L 2202/11

(57) ABSTRACT

An apparatus for cleaning a device is provided which may comprise a chamber for accommodating the device and a carousel disposed within the chamber which comprises a first ultraviolet (UV) light emitting diode (LED) and a second UV LED disposed on the carousel, wherein a first UV light emitted from the first UV LED can at least partially overlap a second UV light emitted from the second UV LED. The carousel can further comprise an air halo disposed on an outer surface of the carousel, the air halo including at least one outlet to expend pressurized air.

### TECHNICAL FIELD

The embodiments described herein relate generally to a cleaning and disinfecting cradle, in particular, to a cleaning and/or disinfecting cradle for virtual reality headsets according to a rotating carousel configuration.

### BACKGROUND

Ultraviolet (UV) light comprising a UV-C wavelength is effective at killing bacteria, viruses, and fungi. UV light with a UV-C wavelength corrupts the DNA of these contagions and can therefore prevent these contagions from replicating. By doing so, the UV light can effectively prevent these bacteria, viruses, and fungi from proliferating as diseases.

The effect of UV light can be maximized when the UV light shines directly on the bacteria, viruses, and fungi. If the UV light is prevented from shining directly on the contagions in all areas, thereby creating shadows of untreated surfaces, then such contagions disposed within these shadows may not be subjected to the DNA corrupting effects of the UV light. As a consequence, these contagions may continue to breed and proliferate as diseases.

A need therefore exists to decrease both the size and number of shadows cast on the surfaces to be cleaned and/or disinfected. Therefore, a need exists for providing methods, systems and devices that effectively treat all surfaces of a product requiring cleaning and/or disinfecting, thereby minimizing or effectively eliminating the presence of shadows within the cleaning and/or disinfecting apparatus. The present disclosure contemplates eliminating and/or minimizing the shadows cast on surfaces to be cleaned and/or disinfected.

### SUMMARY

Aspects of the subject technology relate to an apparatus for disinfecting and/or cleaning a headset. In one aspect, in accordance with various embodiments contemplated herein, an apparatus for disinfecting and cleaning a headset is provided. The apparatus can comprise a chamber for accommodating the headset, and a carousel disposed within the chamber and sized to fit within an inner circumference of the headset. The carousel can comprise a first ultraviolet (UV) light emitting diode (LED) and a second UV LED disposed around a perimeter of the carousel to shine UV light onto the headset. Further, a first UV light emitted from the first UV LED partially can overlap a second UV light emitted from the second UV LED. The carousel can further comprise an air halo disposed on the perimeter of the carousel, the air halo including at least one outlet to blow pressurized air on the headset to remove detritus from surfaces of the headset and dry the headset.

In another aspect, in accordance with various embodiments contemplated herein, an apparatus for cleaning a device is provided. The apparatus can comprise a chamber for accommodating the device and a carousel disposed within the chamber. The carousel can comprise a first ultraviolet (UV) light emitting diode (LED) and a second UV LED disposed on the carousel, wherein a first UV light emitted from the first UV LED can at least partially overlap a second UV light emitted from the second UV LED. The carousel can further comprise an air halo disposed on an outer surface of the carousel, the air halo including at least one outlet to expend pressurized air.

### BRIEF DESCRIPTION OF THE DRAWINGS

The following figures are included to illustrate certain aspects of the embodiments, and should not be viewed as exclusive embodiments. The subject matter disclosed is capable of considerable modifications, alterations, combinations, and equivalents in form and function, as will occur to those skilled in the art and having the benefit of this disclosure.

FIG. 1 depicts a perspective view of a rotating carousel configuration for disinfecting equipment surfaces in accordance with various embodiments.

FIG. 2 depicts a perspective view of a rotating carousel configuration for disinfecting equipment surfaces in accordance with various embodiments.

FIGS. 3A-3C depict example arrangements of light sources in accordance with various embodiments.

FIG. 4 depicts a top view of a pulley system configuration in accordance with various embodiments.

### DETAILED DESCRIPTION

It is to be understood that the present disclosure includes examples of the subject technology and does not limit the scope of the appended claims. Various aspects of the subject technology will now be disclosed according to particular but non-limiting examples. Various embodiments described in the present disclosure may be carried out in different ways and variations, and in accordance with a desired application or implementation. In the following detailed description, numerous specific details are set forth to provide a full understanding of the present disclosure. It will be apparent, however, to one ordinarily skilled in the art that embodiments of the present disclosure may be practiced without some of the specific details. In other instances, well-known structures and techniques have not been shown in detail so as not to obscure the disclosure.

The various embodiments of pivoting carousel configurations for cleaning and/or disinfecting surfaces of a device or piece of equipment (e.g., virtual reality (VR) head mounted displays, VR headsets, etc.) provide a reliable technique for cleaning and/or disinfection by eliminating and/or minimizing shadows cast on the surfaces to be disinfected. The systems disclosed herein are presented in terms of a cleaning and disinfecting cradle for virtual reality headsets according to a pivoting carousel configuration. It will be apparent to those of ordinary skill in the art that the disclosed concepts may be applied to a variety of mechanisms utilizing pivoting carousel configurations for disinfection.

As applicable to any and all embodiments provided herein as well as those reasonably contemplated based on the various embodiments provided herein, the pivoting motion of the carousel can include, but is not limited to, a full rotating motion, a partial rotating motion, a rocking motion, and an oscillating motion. Some of these motion types will be discussed in further detail below. It should be noted that at least any motion facilitated by pivoting about a central point or axis is contemplated, whether specifically identified herein or not.

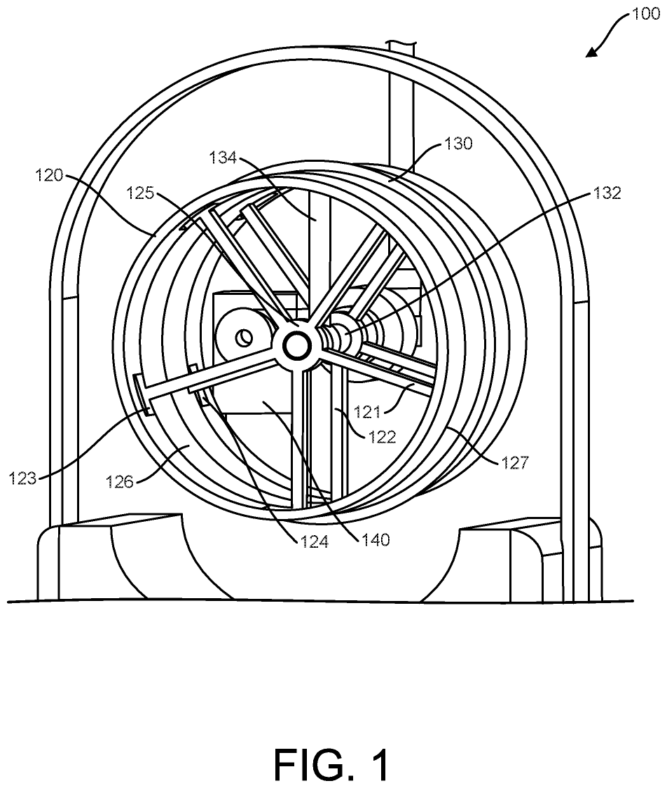

FIG. 1 depicts a perspective view of a pivoting carousel configuration for cleaning and/or disinfecting equipment (or device) surfaces, in accordance with various embodiments. As illustrated in FIG. 1, a pivoting carousel configuration 100 can include a wheel-shaped carousel 120 and an air halo 130. Air halo 130 can be, for example, ring shaped. The carousel can include a first set of spokes 121, a second set of spokes 122, a first row of sockets 123, and a second row of sockets 124. As illustrated in FIG. 1, each spoke can extend from a central axis of the carousel to an inner surface 126 of the carousel. Each socket in the first row of sockets and the second row of sockets can extend from the inner surface 126 of the carousel 120 to an outer surface 128 of the carousel 120. A light source (e.g., UV-C LEDs) printed circuit board (PCB) can be placed in each socket. The number of sockets can vary depending on various factors including, for example, the number of LEDs desired, the power of the LEDs used, and product to be cleansed and/or disinfected. Each product (such as VR headsets) present unique issues as to where shadows can reside and the size and number of those shadows, thus potentially affecting the number and location of sockets needed or desired. In FIG. 1, there is illustrated five sockets in each row of sockets. However, again, that number can vary above and below five for at least the reasons provided above. Moreover, the number of sockets in each row may be an odd number or an even number. The sockets can be arranged equidistant from one another. However, the arrangement of the sockets is not limited to equal distance between the sockets as depicted in FIG. 1.

It should be noted that the vertical cross-section of the carousel is not limited to a circular shape, the full carousel represented by a cylindrical shape as illustrated, for example, in FIG. 1. Though the carousel may be pivoted (as discussed in detail herein), for example, about a central axis, a circular shaped cross-section is not required to allow for such pivoting. Even further, this does not require that the full shape of the carousel by cylindrical as provided, for example, in FIG. 1. In fact, any shaped cross-section with a center point is capable of being pivoted about that center point, extended to a central axis when considering the full carousel structure. To this, the cross-section shape can be, for example and not limited to, a square, oval, square, rectangle, pentagon, octagon, and so on. The full carousel shape can be, for example and not limited to, a cylinder, cone, box, and so one.

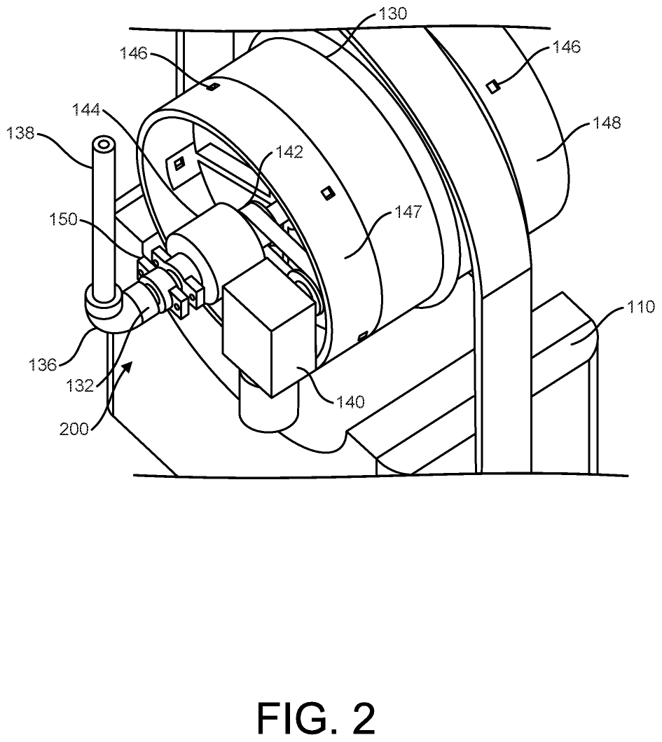

As provided, for example, in FIG. 2, multiple light sources 146 can be arranged in two rows 147/148, in accordance with various embodiments. The arrangement of the two rows 147/148 of light sources 146 can increase the effectiveness of eliminating and/or minimizing the shadow on the surfaces of the equipment (or device) by flooding the exposed surfaces of equipment with light from at least two different angles. The introduction of UV light from more than one angle prevents many shadows from existing on surfaces to be disinfected. Electrical power for the light sources 146 disposed on the PCBs runs from a central air pipe 132 (see FIG. 1) and up each of the spokes 121/122 to the respective light source 146.

Returning to FIG. 1, the air halo 130 can be connected to a central air pipe 132 (or drive shaft) via an air junction pipe 134. The air halo 130 can include air holes (not pictured) that can allow the compressed air traveling through the central air pipe 132 via the air junction pipe 134 to be directed at surfaces of equipment 110 (e.g., VR head mounted displays, VR headsets, etc.) as the carousel 120 pivots. Via this compressed air, the surfaces of the equipment can be blasted with air, thereby removing detritus and drying sweat and oils disposed thereon. The air junction pipe 134 can be affixed to the central air pipe 132 such that junction pipe 134 redirects the flow of air from the central air pipe 132 up to the air halo 130. Referring to FIGS. 1 and 2, for example, the central air pipe 132 can run from the pivoting end of a rotary joint 136 (e.g., 90-degree rotary joint 136) through the center axis (the center axis traveling through central air pipe 132 in the illustrated embodiment) of the first set of light sources 146 of the carousel 120. The first set of light sources 146, as discussed above, can be provided in row 147 in, for example, a wheel shaped configuration as illustrated. Central air pipe 132 can continue forward and terminate at the spokes 121 of the second set of light sources 146 of the carousel 120. Similar to the illustrated two 147, this second set of light sources 146 can be provided in row 148 in, for example, a wheel shaped configuration as illustrated.

The air junction pipe 134 can attach to the central air pipe 132, for example, proximal a halfway point through the carousel 120, allowing air to flow upward to the air halo 130. This assumes that air halo 130 is disposed between the first row of sockets 123 and the second row of sockets 124. In various embodiments, the air halo 130 is disposed between a first UV LED of the first row of sockets 123 and a second UV LED of the second row of sockets 124. Moreover, the air halo 130 need not be positioned at this halfway point as illustrated in FIG. 1. Instead, air halo can be shifted along outer surface 127 as needed depending on many factors including, for example, the length of carousel 120 between the front of carousel 120 (proximate sockets 123) and the rear of carousel 120 (proximate sockets 124), or the type of equipment being cleansed and/or disinfected.

The carousel 120 can further include one or more restraining screw recesses 125 for affixing the carousel 120 to the central air pipe 132. The restraining screw recesses 125 are fitted through hubs (two hubs may be present) of the carousel 120.

Returning to FIG. 2, FIG. 2 depicts a perspective view of a pivoting carousel 120 configuration for disinfecting equipment surfaces, in accordance with various embodiments. The pivoting carousel configuration can further include a pulley system 200, which can include a carousel motor 140, a pulley belt 142, a slip ring 144, a set of wall retaining rings 150 (or mounting rings), a rotary joint 136 (e.g., a 90-degree rotary joint), and a stationary air feed line 138. The carousel motor 140, pulley belt 142 and slip ring 144 together can form the pulley system 200 configured to pivot carousel 120 via central air pipe 132 (or drive shaft).

The slip ring 144, in accordance with various embodiments, can be considered a mechanism that prevents the wires attached to the UVC lights from twisting as the carousel pivots.

Retaining rings 150 (or mounting rings) are features that can allow the central air pipe 132 (or drive Shaft) to pivot while the rings themselves are mounted to supporting walls of system 100 such that carousel 120 (and associated components discussed herein) is supported so that it hangs properly inside the equipment to be cleansed and/or disinfected.

The carousel motor 140 can include a pulley gear (not pictured) attached thereto. The carousel motor can be relatively small. The carousel motor 140 can turn the pulley gear to drive the pulley belt 142. The pulley belt 142 can be attached to the slip ring 144. When the carousel motor 140 turns the pulley gear, the pulley belt 142 can translate the drive of the pulley gear to the slip ring 144. The slip ring 144 can then turn the central air pipe 132. The carousel 120 thereby pivots as the central air pipe 132 is turned.

The rotating carousel configuration can be housed in a cradle that houses the equipment 110 while the equipment 110 is being cleansed and/or disinfected by the rotating carousel apparatus 120. An air source (no pictured), for supplying air that can flow through the air feed line 138 into the central air pipe 132, can reside outside the cradle. One of the wall retaining rings 150 (e.g., proximate the rotary joint 136) can be attached to an outer surface of a back wall of the cradle (not illustrated), and another one of the wall retaining rings 150 (e.g., proximate the slip ring 144) can be attached to an inner surface of the back wall of the cradle (not illustrated). Though not illustrated, it is reasonable to contemplate a back wall residing between the set of retaining rings 150 illustrated in FIG. 2.

As provided by the example embodiment illustrated in FIG. 2, for example, the central air pipe 132 can travel through the wall retaining rings 150 and the back wall of the cradle (not illustrated). The wall retaining rings 150 can hold the central air pipe 132 in place, thereby supporting the weight of the carousel 120 and keeping the central air pipe 132 suspended (e.g., about a central axis relative to carousel 120) using the rotary joint 136 (e.g., 90-degree rotary joint 136) out and away from the back wall of the cradle (not illustrated) that houses the equipment and the rotating carousel 120. The wall retaining rings 150 can hold the pipe tightly in place by restraining screws 125, but the central air pipe 132 may still freely pivot within the wall retaining rings 150. The stationary air feed line 138 can allow compressed air from the air source (not pictured) to flow into the central air pipe 132 via the rotary joint 136 (e.g., 90-degree rotary joint 136).

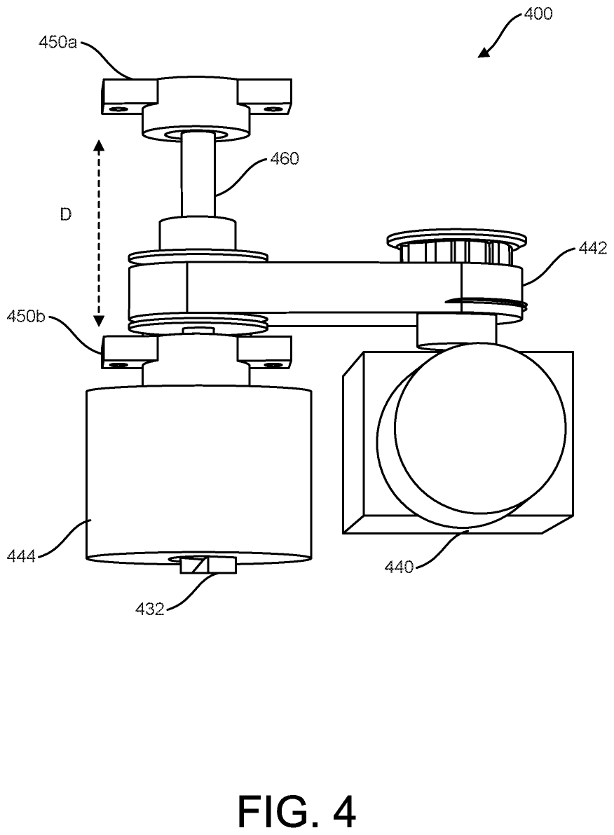

Referring to FIG. 4, a pulley system 400 is provided, in accordance with various embodiments. Similar to the example pulley system 200 illustrated in FIG. 2, pulley system 400 can include a carousel motor 440, a pulley belt 442, a slip ring 444, and a set of wall retaining rings 450a/b (or mounting rings). The carousel motor 440, pulley belt 442 and slip ring 444 together can form the pulley system 400 configured to pivot carousel 120 (see FIG. 1 for example) via a central air pipe 432 (or drive shaft). In pulley system 400, as compared to pulley system 200 illustrated in FIG. 2, pulley system 400 can be configured such that pulley belt 442 resides outside the interior of the carousel (see, by comparison, pulley belt 142 illustrated in FIG. 2). One example technique to provide for this pulley belt 442 location is by providing a separation distance D between retaining rings 450a and 450b. Such a separation distance can allow for the presence of both pulley belt 442 and a back wall (not pictured) that could intersect at least a portion of a retaining ring bar 460 such that retaining ring 450a could be located exterior to the back wall while the remaining components of pulley system 400 could be located interior to the back wall.

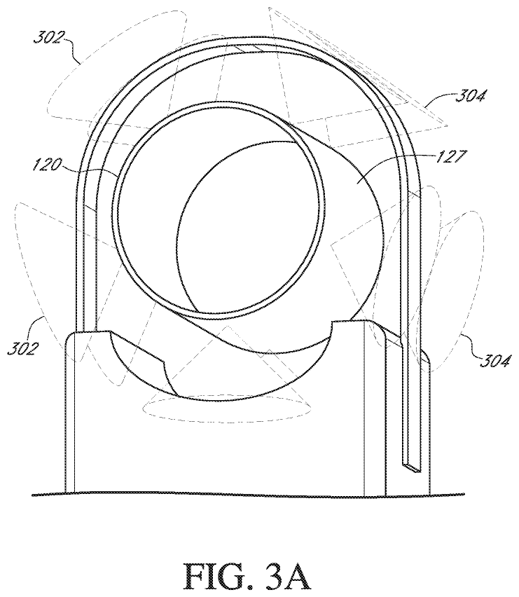

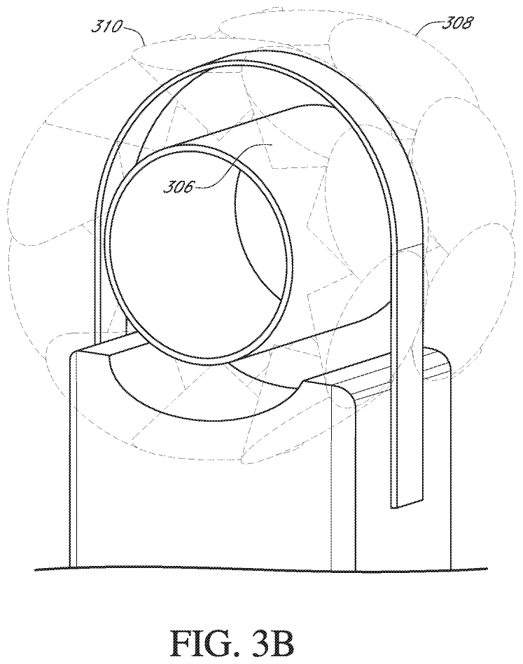

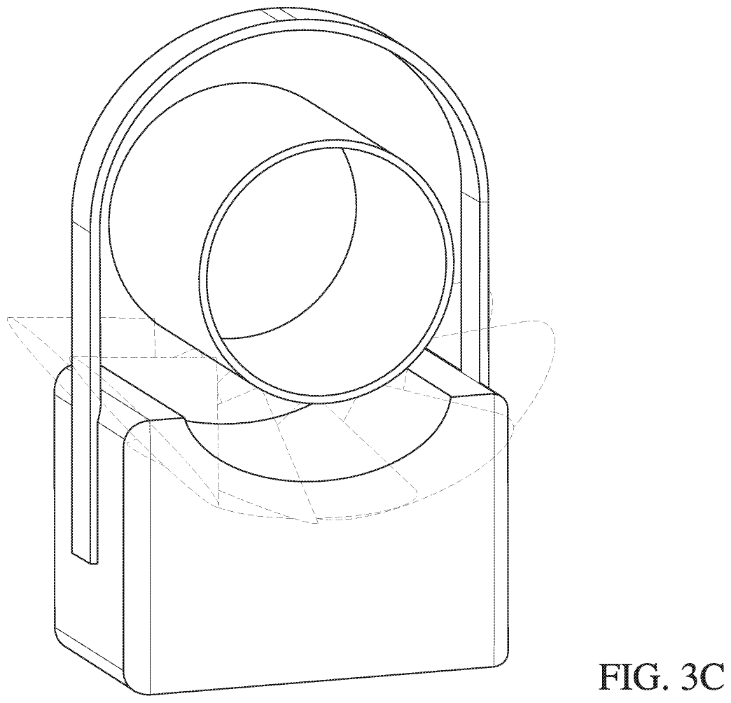

FIGS. 3A-3C conceptually depict example arrangements of light sources in accordance with various embodiments. The light sources (e.g., UV-C LEDs) are disposed on the PCBs so that the light from the light sources projects from the exterior surface 127 of the carousel 120. As illustrated by the various example illustrations of FIGS. 3A-3C, the light emitted from at least one light source can overlap light from another light source. For example, the light emitted from at least one light source in the first row of sockets (see FIGS. 1 and 2 for example) can overlap light emitted from at least one light source in the second row of sockets (see FIGS. 1 and 2 for example). This can create an overlapping cone effect. As illustrated in FIG. 3A, a first set of UV LEDs 302 and a second set of UV LEDs 304 are disposed around a perimeter of the carousel 120 to shine UV light onto the headset. The LEDs can be positioned such that the first row of sockets (see FIGS. 1 and 2 for example) includes the at least one of the first set of UV LEDs 302 and the second row of sockets (see FIGS. 1 and 2 for example) includes at least one of the second set of UV LEDs 304. The light emitted from at least one of the first set of UV LEDs 302 can be configured and positioned to partially overlap the light emitted from at least one of the second UV LEDs 304.

As illustrated in FIG. 3B, in accordance with various embodiments, the light emitted from a light source (e.g., UV-C LED) can be positioned to partially overlap both the light from an adjacent light source in the same row and the light from a light source in the other row. For example, the first row of sockets (see FIGS. 1 and 2 for example) can include a first UV LED 306 and a third UV LED 310, and the second row of sockets (see FIGS. 1 and 2 for example) can include a second UV LED 308. The UV light emitted from the first UV LED 306 can thus be positioned to partially overlap the UV light emitted from the second UV LED 308 and UV light emitted from the third UV LED 310.

Similarly, FIG. 3C illustrates an example light source configuration whereby the light from a light source partially overlap the light from adjacent light sources in the same row and the light from a light source in the other row.

It should be understood that the light sources can be positioned in various ways as needed to provide the necessary UV light coverage necessary to clean and/or disinfect the specific equipment involved. For example, light sources can be configured in rows as illustrated in FIGS. 3A-3C. One or more of those light sources can be positioned such that its emitted light overlaps light emitted from another light source on its same row, light emitted from another light source on another row, a combination of both overlapping configurations, or no overlap at all. It should be understood that light sources do not need to be provided in rows. Moreover, if the light sources are associated with specific sockets on the carousel, the sockets themselves need not be provided in rows. Instead, the sockets and/or associated light sources can have any necessary configuration, even including a random distribution, to provide the necessary light coverage. Furthermore, even if sockets are provided in rows as is illustrated, a light source does not need to be provided in conjunction with each socket. As a result, there may be less light sources than sockets. On the other hand, as needed, more light sources can be provided than sockets provided.

In operation, the equipment (e.g., headset) may be positioned (e.g., in a cradle-not pictured) so that the pivoting carousel 120 fits within the inner circumference of the headset, thereby positioning the UV-C LEDs on the carousel 120 a specific distance from the equipment surface. For example, the UV-C LEDs can be positioned within two inches or less of the headset surfaces. This can be facilitated, as discussed above, via retaining rings 150 (or mounting rings) such as is discussed above, in accordance with various embodiments.

Further in operation, the flow of compressed air, when initiated, proceeds through the stationery air feed line 138 to the air halo 130 via the joint 136 (e.g., 90-degree rotary joint 136), the central air pipe 132, and the air junction pipe 134. The carousel 120 pivots (see above) about the central air pipe 132 continuously throughout the disinfection cycle to reduce the potential for shadows casting on the surfaces of the headset. When pivoting in a rotational mode, the rotational speed may be based on the length of time required for each surface to be exposed to direct UV-C light to eliminate contagions. In accordance with various embodiments, the arrangement of light sources (e.g., arrangements in FIGS. 3A-3C) allow the surfaces of the headset to be exposed continuously to direct UV-C. In such cases, the speed may be preset based on the efficiency and mechanical considerations. Cycle time may be extended to enable sufficient exposure time on all potential target surfaces on the equipment to corrupt the contagions thereon and prevent said contagions from remaining and potentially replicating.

The equipment (e.g., headset) may then be subjected to air jet blasts through the vents of the air halo, which may knock free remaining detritus and sweat of previous users from the headset surfaces, and dry the surfaces so the headset surfaces are disinfected and ready for use by the next user.

As the carousel 120 pivots, light cones emitted by the UV-C light sources sweep across the surfaces to be disinfected thereby changing the direction of exposure of light on each surface. This change in the direction of incident light, combined with the overlapping cones of light as described hereinabove, considerable reduces the potential for shadows on irregular surfaces. Some irregular surfaces for which this method/system improves disinfection include, for example, hook-and-loop fastener (Velcro®), uneven and/or roughened materials, and/or materials having crevices. Even in the cases of irregular surfaces, the various embodiments for light sweeping operates to expose all surfaces to the ultraviolet light during the disinfection cycle.

As discussed above, the carousel 120 can pivot as necessary to emit UVC light to all necessary services of the equipment being treated. As such, the overall carousel may rotate 360 degrees or any value less than 360 degrees. In accordance with various embodiments, the carousel can be configured to rock or oscillate back and forth, rotating approximately 20 to about 30 degrees in a direction before reversing direction and going back rotate the other direction. In accordance with various embodiments, the carousel can be configured to rock or oscillate between about 5 to about 10 degrees, about 10 to about 15 degrees, about 15 to about 20 degrees, about 20 to about 25 degrees, about 25 to about 30 degrees, about 30 to about 35 degrees, about 35 to about 40 degrees, about 40 to about 45 degrees, and any range using any of these values. Less than full rotation can be of particular benefit when, for example, UVC lights are positioned on the carousel such that full or nearly full rotation is not necessary in order to get emitted lights to all surfaces of interest of the equipment being treated.

In accordance with various embodiments contemplated herein, an apparatus for disinfecting and cleaning a headset is provided. The apparatus can comprise a chamber for accommodating the headset, and a carousel disposed within the chamber and sized to fit within an inner circumference of the headset. The carousel can comprise a first ultraviolet (UV) light emitting diode (LED) and a second UV LED disposed around a perimeter of the carousel to shine UV light onto the headset. Further, a first UV light emitted from the first UV LED partially can overlap a second UV light emitted from the second UV LED. The carousel can further comprise an air halo disposed on the perimeter of the carousel, the air halo including at least one outlet to blow pressurized air on the headset to remove detritus from surfaces of the headset and dry the headset.

The carousel can further comprise a first row of UV LEDs and a second row of UV LEDs, wherein the first row of UV LEDs comprises the first UV LED, and wherein the second row of UV LEDs comprises the second UV LED.

The carousel can further comprise a third UV LED, wherein the first row of UV LEDs comprises the first UV LED and the third UV LED, and wherein the first UV light emitted from the first UV LED partially overlaps the second UV light emitted from the second UV LED and a third UV light emitted from the third UV LED.

The air halo can be disposed between the first row of UV LEDs and the second row of UV LEDs. Alternatively, the air halo can be disposed between the first UV LED and the second UV LED.

The carousel can further comprise a plurality of sockets for receiving the first UV LED and the second UV LED, wherein each of the sockets extends from an inner surface of the carousel to an outer surface of the carousel and receives the UV LED to allow light from the UV LED to emit from the outer surface of the carousel.

The inner circumference of the headset can be defined by an open area between a surface of the headset configured for placement against a face of a user and straps or arms of the headset configured to secure the headset around a head of the user.

The apparatus can further comprise a rotating mechanism for rotating the carousel, wherein the first UV LED and the second UV LED emit the UV light while the rotating mechanism rotates the carousel.

The at least one outlet of the air halo can be positioned to blow directly at a portion of the surface of the headset configured for placement against a face of the user. The air halo can comprise a plurality of outlets. The air halo can extend substantially the entire perimeter of the carousel.

The carousel can further comprise an air pipe disposed on a central axis of the carousel, and at least one spoke connecting the carousel to the air pipe. The at least one spoke can connect the air halo to the air pipe. The air halo can be a tube, with the at least one outlet provided on the tube. A plurality of outlets can be provided on the tube. The tube can extend substantially the entire perimeter of the carousel.

In accordance with various embodiments contemplated herein, an apparatus for cleaning a device is provided. The apparatus can comprise a chamber for accommodating the device and a carousel disposed within the chamber. The carousel can comprise a first ultraviolet (UV) light emitting diode (LED) and a second UV LED disposed on the carousel, wherein a first UV light emitted from the first UV LED can at least partially overlap a second UV light emitted from the second UV LED. The carousel can further comprise an air halo disposed on an outer surface of the carousel, the air halo including at least one outlet to expend pressurized air.

The carousel can further comprise a drive shaft connected to the carousel and positioned about an interior axis of the carousel, the drive shaft configured to pivot the carousel. The apparatus can further comprise a carousel motor connected to the drive shaft, the motor configured to power the drive shaft to rotate the carousel between 5 and 45 degrees in a clockwise and counterclockwise direction. The apparatus can further comprise a carousel motor connected to the drive shaft, the motor configured to power the drive shaft to rotate the carousel more than 45 degrees in a clockwise and counterclockwise direction. The apparatus can further comprise a carousel motor connected to the drive shaft, the motor configured to power the drive shaft to rotate the carousel at least 360 degrees in a single direction. The drive shaft is positioned about a central axis of the carousel.

The carousel can further comprise a first row of UV LEDs and a second row of UV LEDs, wherein the first row of UV LEDs comprises the first UV LED, and wherein the second row of UV LEDs comprises the second UV LED. The carousel can further comprise a third UV LED, wherein the first row of UV LEDs comprises the first UV LED and the third UV LED, and wherein the first UV light emitted from the first UV LED partially overlaps the second UV light emitted from the second UV LED and a third UV light emitted from the third UV LED.

The air halo can be disposed between the first row of UV LEDs and the second row of UV LEDs. Alternatively, the air halo can be disposed between the first UV LED and the second UV LED.

The carousel can further comprise a plurality of sockets for receiving the first UV LED and the second UV LED, wherein each of the sockets extends from an inner surface of the carousel to an outer surface of the carousel and receives the respective UV LED to allow light from the UV LED to emit from the outer surface of the carousel.

The device can be a headset, the headset can define an inner circumference, the carousel can be sized to fit within the inner circumference of the headset, and the inner circumference of the headset can be defined by an open area between a surface of the headset configured for placement against a face of a user and straps or arms of the headset configured to secure the headset around a head of the user.

The at least one outlet of the air halo can be positioned to expend directly at a portion of a surface of the device. The carousel can further comprise at least one spoke connecting the carousel to the drive shaft. The at least one spoke can connect the air halo to the drive shaft. The air halo can comprise a plurality of outlets. The air halo can extend substantially the outer surface of the carousel.

The air halo can be a tube, with the at least one outlet provided on the tube. A plurality of outlets are provided on the tube. The tube can extend substantially the entire outer surface of the carousel.

In accordance with various embodiments herein, a device that is relatively simple in application and prevents contagions (e.g., viruses, bacteria, and fungi) from being protected by shadows during disinfection of target surfaces by UV-C light represents an advancement in the art. The reduction of shadows present on objects to be disinfected, even when the object and/or the surfaces thereof are irregular, can increase the effectiveness of the cleaning and disinfecting cradle.

The following represent example advantages/applications associated with the various embodiments herein.

The inventors have discovered that the various embodiments herein may be advantageous because minimal heat is produced as a byproduct of the disinfecting processes associated with the various system and apparatus embodiments herein, no heat is actively used for a disinfecting function, no liquid is produced (e.g., requiring disposal), and no waste is produced. As a result, inventors have discovered techniques to achieve optimal disinfection while providing a “green”, i.e., eco-friendly. These advantages are realized, for example, because the various embodiments herein implement features such as UV-C LEDs, nano and compressed air blasts, and specific orientation of light sources and pulley systems to achieve efficient disinfection of various equipment types.

The various embodiments herein may be useful in research and development applications. The various embodiments herein may further be useful for virtual and/or augmented reality headsets, haptic feedback equipment, remote controllers, game controllers, gun-like controllers, and/or other equipment associated therewith.

The various embodiments herein may represent advantages over conventional “room zap” disinfection wherein zapping an entire cabinet/room full of supplies in one, relatively long cycle (longer than 1 minute) is performed during an end-of-day cleaning cycle for disinfecting all equipment located in situ. By comparison, the various embodiments provide for repeated disinfecting of equipment such that user-to-user infection (e.g., where multiple uses of equipment occur on a daily basis) is severely minimized

The various embodiments herein may be used to disinfect boxes/containers of stethoscopes, boxes/containers for ad hoc small medical supplies, boxes/containers for ad hoc miscellaneous sanitation, and/or boxes/containers for hospital, ambulance, and/or emergency services.

Further, the various embodiments herein may be applied to the disinfection of hard hats, hard hats with glasses, helmets for football players, helmets for use with bikes and/or scooters, and/or other suitable types of protective headwear.

The various embodiments herein may also be applied to the disinfection of remote controls for electronics, video gaming console controls, wands (such as for metal detection), handheld electronics, mobile devices, mobile devices with associated audio transducers, pens, pencils, keys and/or keychains, eyeglasses, sunglasses, safety glasses (such as for construction, automotive racing, and/or other sport or work protection) headphones, airplane headphones, headphones for audio tours, microphones, microphone headsets, and/or other suitable devices.

The various embodiments herein may be applied to special supplies for children in hospitals and/or boxes for cars (such as may be disposed within a back of a car seat for children's′ toys, TVs, audio/visual devices, etc.)

Still further, the various embodiments herein may be applied to disinfecting dental supplies and/or as a separate UV light that is applied to electric toothbrush heads.

The various embodiments herein may also be applied to quick cycle boxes in police stations for disinfecting finger printing devices, etc. and/or other public use hardware such as handcuffs, restraints, etc.

Further, the various embodiments herein may be applied to children's toy boxes and may include one or more safety mechanisms.

Additionally, the various embodiments herein may be applied to the disinfecting of baseball bats, footballs, softballs, baseball and softball mits/gloves, baseballs, mouth guards, boxing gloves, MMA gloves, weightlifting gloves, exercise mats, knee and elbow pads, sleeves, braces, and/or other suitable sport and fitness equipment. The system/method may be applied as a drying and decontamination unit for team sports gear.

The various embodiments herein may also be applied to breathing devices, cosmetics devices, cosmetics applicators, SCUBA respirators, CPAP (Continuous positive airway pressure) devices, inhalers, breathalyzers, and/or other such suitable devices. The various embodiments herein may be applied to medical devices that cannot be exposed to heat and/or liquids, but for which disinfection is still desired.

The above-described embodiments of the invention are presented for purposes of illustration and not of limitation. While these embodiments of the invention have been described with reference to numerous specific details, one of ordinary skill in the art will recognize that the invention can be embodied in other specific forms without departing from the spirit of the invention. Thus, one of ordinary skill in the art would understand that the invention is not to be limited by the foregoing illustrative details, but rather is to be defined by the appended claim.

### Recitation of Some Embodiments of the Disclosure

1. An apparatus for disinfecting and cleaning a headset, the apparatus comprising: a chamber for accommodating the headset; and a carousel disposed within the chamber and sized to fit within an inner circumference of the headset, the carousel comprising: a first ultraviolet (UV) light emitting diode (LED) and a second UV LED disposed around a perimeter of the carousel to shine UV light onto the headset, wherein a first UV light emitted from the first UV LED partially overlaps a second UV light emitted from the second UV LED; and an air halo disposed on the perimeter of the carousel, the air halo including at least one outlet to blow pressurized air on the headset to remove detritus from surfaces of the headset and dry the headset.

2. The apparatus of Embodiment 1, wherein the carousel comprises a first row of UV LEDs and a second row of UV LEDs, wherein the first row of UV LEDs comprises the first UV LED, and wherein the second row of UV LEDs comprises the second UV LED.

3. The apparatus of Embodiment 1 or 2, wherein the carousel comprises a third UV LED, wherein the first row of UV LEDs comprises the first UV LED and the third UV LED, and wherein the first UV light emitted from the first UV LED partially overlaps the second UV light emitted from the second UV LED and a third UV light emitted from the third UV LED.

4. The apparatus of Embodiment 1 or 2, wherein the air halo is disposed between the first row of UV LEDs and the second row of UV LEDs.

5. The apparatus of Embodiment 1, wherein the air halo is disposed between the first UV LED and the second UV LED.

6. The apparatus of Embodiment 1, the carousel further comprising a plurality of sockets for receiving the first UV LED and the second UV LED, wherein each of the sockets extends from an inner surface of the carousel to an outer surface of the carousel and receives the UV LED to allow light from the UV LED to emit from the outer surface of the carousel.

7. The apparatus of Embodiment 1 or 6, wherein the inner circumference of the headset is defined by an open area between a surface of the headset configured for placement against a face of a user and straps or arms of the headset configured to secure the headset around a head of the user.

8. The apparatus of Embodiment 1, further comprising a rotating mechanism for rotating the carousel, wherein the first UV LED and the second UV LED emit the UV light while the rotating mechanism rotates the carousel.

9. The apparatus of Embodiment 1, wherein the at least one outlet of the air halo is positioned to blow directly at a portion of the surface of the headset configured for placement against a face of the user.

10. The apparatus of Embodiment 1, the carousel further comprising: an air pipe disposed on a central axis of the carousel; and at least one spoke connecting the carousel to the air pipe.

11. The apparatus of Embodiment 1 or 10, wherein the at least one spoke connects the air halo to the air pipe.

12. The apparatus of Embodiment 1, wherein the air halo is a tube, with the at least one outlet provided on the tube.

13. The apparatus of Embodiment 1 or 12, wherein a plurality of outlets are provided on the tube.

14. The apparatus of Embodiment 1, wherein the air halo comprises a plurality of outlets.

15. The apparatus of Embodiment 1 or 12, wherein the tube extends substantially the entire perimeter of the carousel.

16. The apparatus of Embodiment 1, wherein the air halo extends substantially the entire perimeter of the carousel.

17. An apparatus for cleaning a device, the apparatus comprising: a chamber for accommodating the device; a carousel disposed within the chamber, the carousel comprising: a first ultraviolet (UV) light emitting diode (LED) and a second UV LED disposed on the carousel, wherein a first UV light emitted from the first UV LED at least partially overlaps a second UV light emitted from the second UV LED; and an air halo disposed on an outer surface of the carousel, the air halo including at least one outlet to expend pressurized air.

18. The apparatus of Embodiment 17, the carousel further comprising a drive shaft connected to the carousel and positioned about an interior axis of the carousel, the drive shaft configured to pivot the carousel.

19. The apparatus of Embodiment 18, further comprising a carousel motor connected to the drive shaft, the motor configured to power the drive shaft to rotate the carousel between 5 and 45 degrees in a clockwise and counterclockwise direction.

20. The apparatus of Embodiment 18, further comprising a carousel motor connected to the drive shaft, the motor configured to power the drive shaft to rotate the carousel more than 45 degrees in a clockwise and counterclockwise direction.

21. The apparatus of Embodiment 18, further comprising a carousel motor connected to the drive shaft, the motor configured to power the drive shaft to rotate the carousel at least 360 degrees in a single direction.

22. The apparatus of Embodiment 18, wherein the drive shaft is positioned about a central axis of the carousel.

23. The apparatus of any of Embodiments 17 to 22, the carousel further comprising a first row of UV LEDs and a second row of UV LEDs, wherein the first row of UV LEDs comprises the first UV LED, and wherein the second row of UV LEDs comprises the second UV LED.

24. The apparatus of Embodiment 23, the carousel further comprising a third UV LED, wherein the first row of UV LEDs comprises the first UV LED and the third UV LED, and wherein the first UV light emitted from the first UV LED partially overlaps the second UV light emitted from the second UV LED and a third UV light emitted from the third UV LED.

25. The apparatus of Embodiment 23, wherein the air halo is disposed between the first row of UV LEDs and the second row of UV LEDs.

26. The apparatus of any of Embodiments 17 to 25, wherein the air halo is disposed between the first UV LED and the second UV LED.

27. The apparatus of any of Embodiments 17 to 26, the carousel further comprising a plurality of sockets for receiving the first UV LED and the second UV LED, wherein each of the sockets extends from an inner surface of the carousel to an outer surface of the carousel and receives the respective UV LED to allow light from the UV LED to emit from the outer surface of the carousel.

28. The apparatus of any of Embodiments 17 to 27, wherein the device is a headset, the headset defines an inner circumference, the carousel is sized to fit within the inner circumference of the headset, and wherein the inner circumference of the headset is defined by an open area between a surface of the headset configured for placement against a face of a user and straps or arms of the headset configured to secure the headset around a head of the user.

29. The apparatus of any of Embodiments 17 to 28, wherein the at least one outlet of the air halo is positioned to expend directly at a portion of a surface of the device.

30. The apparatus of and of Embodiments 18 to 29, the carousel further comprising at least one spoke connecting the carousel to the drive shaft.

31. The apparatus of Embodiment 30, wherein the at least one spoke connects the air halo to the drive shaft.

32. The apparatus of any of Embodiments 17 to 31, wherein the air halo is a tube, with the at least one outlet provided on the tube.

33. The apparatus of Embodiment 32, wherein a plurality of outlets are provided on the tube.

34. The apparatus of any of Embodiments 17 to 33, wherein the air halo comprises a plurality of outlets.

35. The apparatus of Embodiment 34, wherein the tube extends substantially the entire outer surface of the carousel.

36. The apparatus of any of Embodiments 17 to 35, wherein the air halo extends substantially the outer surface of the carousel.

37. The apparatus of any of Embodiments 1 to 36, wherein at least one UV LED emits UV-C light.

1-16. (canceled)

17. An apparatus for cleaning a device, the apparatus comprising:
a chamber for accommodating the device; and
a carousel disposed within the chamber, the carousel comprising:
a first ultraviolet (UV) light emitting diode (LED) and a second UV LED disposed on the carousel, wherein a first UV light emitted from the first UV LED at least partially overlaps a second UV light emitted from the second UV LED; and
an air halo disposed on an outer surface of the carousel, the air halo including at least one outlet to expend pressurized air.

18. The apparatus of claim 17, the carousel further comprising a drive shaft connected to the carousel and positioned about an interior axis of the carousel, the drive shaft configured to pivot the carousel.

19. The apparatus of claim 18, further comprising a carousel motor connected to the drive shaft, the motor configured to power the drive shaft to rotate the carousel between 5 and 45 degrees in a clockwise and counterclockwise direction.

20. The apparatus of claim 18, further comprising a carousel motor connected to the drive shaft, the motor configured to power the drive shaft to rotate the carousel more than 45 degrees in a clockwise and counterclockwise direction.

21. The apparatus of claim 18, further comprising a carousel motor connected to the drive shaft, the motor configured to power the drive shaft to rotate the carousel at least 360 degrees in a single direction.

22. The apparatus of claim 18, wherein the drive shaft is positioned about a central axis of the carousel.

23. The apparatus of claim 17, the carousel further comprising a first row of UV LEDs and a second row of UV LEDs, wherein the first row of UV LEDs comprises the first UV LED, and wherein the second row of UV LEDs comprises the second UV LED.

24. The apparatus of claim 23, the carousel further comprising a third UV LED, wherein the first row of UV LEDs comprises the first UV LED and the third UV LED, and wherein the first UV light emitted from the first UV LED partially overlaps the second UV light emitted from the second UV LED and a third UV light emitted from the third UV LED.

25. The apparatus of claim 23, wherein the air halo is disposed between the first row of UV LEDs and the second row of UV LEDs.

26. The apparatus of claim 17, wherein the air halo is disposed between the first UV LED and the second UV LED.

27. The apparatus of claim 17, the carousel further comprising a plurality of sockets for receiving the first UV LED and the second UV LED, wherein each of the sockets extends from an inner surface of the carousel to an outer surface of the carousel and receives the respective UV LED to allow light from the UV LED to emit from the outer surface of the carousel.

28. The apparatus of claim 17, wherein the device is a headset, the headset defines an inner circumference, the carousel is sized to fit within the inner circumference of the headset, and wherein the inner circumference of the headset is defined by an open area between a surface of the headset configured for placement against a face of a user and straps or arms of the headset configured to secure the headset around a head of the user.

29. The apparatus of claim 17, wherein the at least one outlet of the air halo is positioned to expend directly at a portion of a surface of the device.

30. The apparatus of claim 18, the carousel further comprising at least one spoke connecting the carousel to the drive shaft.

31. The apparatus of claim 30, wherein the at least one spoke connects the air halo to the drive shaft.

32. The apparatus of claim 17, wherein the air halo is a tube, with the at least one outlet provided on the tube.

33. The apparatus of claim 32, wherein a plurality of outlets are provided on the tube.

34. The apparatus of claim 17, wherein the air halo comprises a plurality of outlets.

35. (canceled)

36. The apparatus of claim 17, wherein the air halo extends substantially the outer surface of the carousel.

37. The apparatus of claim 17, wherein at least one UV LED emits UV-C light.
